(19) United States
(12) Patent Application Publication
(10) Pub. No.: US 2007/0031333 A1
(43) Pub. Date: Feb. 8, 2007

(54) Novel F-18 labeled annexin V, synthesis thereof, and use thereof

(76) Inventors: Ronnie C. Mease, Fairfax, VA (US); Jeffrey A. Toretsky, Silver Spring, MD (US); Irving N. Weinberg, Bethesda, MD (US)

Correspondence Address: PATENT ADMINISTRATOR;KATTEN MUCHIN ROSENMAN LLP, 1025 THOMAS JEFFERSON STREET, N.W., EAST LOBBY: SUITE 700, WASHINGTON, DC, 20007-5201, US

(21) Appl. No.: 10/740,688

(22) Filed: Dec. 22, 2003

Related U.S. Application Data
(60) Provisional application No. 60/437,719, filed on Jan. 2, 2003.

(51) Int. Cl. A61K 51/00

(52) U.S. Cl. 424/1.69

(57) ABSTRACT

A method for noninvasive measurement of apoptosis is described. The method includes the steps of labeling Annexin V with a positron emitter, injecting the labeled Annexin V into a target cell group, obtaining an image of the target cell group using a positron emission tomography scanner, and evaluating the image to determine an amount of cell death within the target cell group. The target cell group may be a lesion or a suspected tumor. The positron emitter may be F-18. The step of labeling Annexin V with F-18 may include the steps of selecting an F-18 labeled small molecule containing a protein conjugating group, synthesizing and purifying the selected molecule, producing a high specific activity prosthetic group as a result of the synthesizing and purifying step, and conjugating the prosthetic group to the Annexin V. The produced prosthetic group may be N-succinimidyl-4-[¹⁸F]fluorobenzoate (SFB).

### CROSS-REFERENCE TO RELATED APPLICATIONS

This application claims priority under 35 U.S.C. §119(e) to U.S. Provisional Application Ser. No. 60/437,719, entitled “Novel F-18 Labeled Annexin V, Synthesis Thereof, and Use Thereof”, filed Jan. 2, 2003, the contents of which are incorporated by reference herein.

### BACKGROUND OF THE INVENTION

1. Field of the Invention

This invention disclosure addresses development of new imaging technology for noninvasive quantitative measurement of apoptosis in vivo. Apoptosis is genetically defined cell death. Understanding the role of apoptosis is critical to the development of new molecular-based therapeutic modalities. An apoptosis-imaging agent could be used to modify therapy for cancer patients, and to monitor cardiac transplant patients. Annexin V is a protein that binds to exposed phosphatidylserine on dead or dying cells. This invention disclosure describes a method to label Annexin-V with a positron emitter (F-18) appropriate for PET scanning. Potential advantages of PET include improved spatial resolution and quantitation, which will be important for monitoring early cancers and for cardiac studies.

2. Description of the Related Art

Cancer cells survive, in part, due to specific signaling pathways that prevent apoptosis in cells harboring protooncogene or suppressor oncogene mutations. A current goal of cancer research is to develop novel therapeutics that will specifically target mutated gene products that are responsible for cancer cell growth and resistance to apoptosis. Thus, many new anti-tumor agents block intracellular survival signaling pathways which then permits apoptotic cell death. Since current chemotherapy dosages are based on the maximally tolerated dose, both novel therapeutics and current chemotherapy agents would benefit from direct measure of tumor cell kill to achieve the biologic goal without causing excess toxicity.

Apoptosis is a genetically defined cell death that involves activation of a core enzymatic machine consisting of cysteine proteases, called caspases. An early molecular event in apoptosis is the flipping of phosphatidylserine (PS) from the inner lipid bilayer to the outer layer of the plasma membrane. Annexin V binds PS with extremely high affinity (Kd=7 nmol/L), as well as specificity, and has been used in vitro to identify apoptotic cells. Technetium-99m labeled Annexin V (Apomate™) is currently in early clinical trials for the detection of apoptosis by Single Photon Emission Computed Tomography (SPECT) in cardiac allograft rejection. Apomate™ as a marker of tumor cell death is also being explored.

In Positron Emission Tomography (PET), the simultaneous detection of the two coincident 511 keV gamma rays from the annihilation of a positron permits greater sensitivity and spatial resolution than that obtained with SPECT and provides truly quantitative imaging. Annexin V labeled with the positron emitter F-18 will be capable of detecting tumor cell death, thereby serving as an early predictor of clinical response to anti-tumor therapy. It is believed that the inherent advantages of PET would make ¹⁸F-Annexin V a better imaging agent than Apomate™ for quantifying the amount of cell death in tumors, especially in small metastatic lesions.

Traditional anti-cancer drug development focused on agents that directly killed cells; thus end-points of clinical trials required tumor regression. New anti-cancer therapies target specific molecular pathways. These molecular targeting agents are used to amplify the effect of other anti-cancer drugs. Investigators who are interested in new drug development need to determine whether a novel agent affects the target in a specified fashion. Traditional tests of novel agents measure tumor regression 6 to 12 weeks following the therapy. Tumor regression with this time frame is the result of some combination of increased cell death and decreased cancer growth. It is therefore difficult with traditional regression techniques to assess the effect of cell death alone on tumor response. Early response detection methods are therefore needed in order to determine the effectiveness of novel anti-tumor agents.

### SUMMARY OF THE INVENTION

The invention provides a method for noninvasive measurement of apoptosis. The method includes the steps of labeling Annexin V with a positron emitter, injecting the labeled Annexin V into a target cell group, obtaining an image of the target cell group using a positron emission tomography scanner, and evaluating the image to determine an amount of cell death within the target cell group. The target cell group may be a lesion or a suspected tumor. The positron emitter may be F-18.

The step of labeling Annexin V with F-18 may include the steps of selecting an F-18 labeled small molecule containing a protein conjugating group, synthesizing and purifying the selected molecule, producing a high specific activity prosthetic group as a result of the synthesizing and purifying step, and conjugating the prosthetic group to the Annexin V. The produced prosthetic group may be one of 4-[¹⁸F]fluorophenacyl-bromide (FPB), N-succinimidyl-8-[(4′-[¹⁸F]fluorobenzyl)amino] suberate (SFBS), or N-succinimidyl-4-[¹⁸F]fluorobenzoate (SFB). When the produced prosthetic group is SFB, the step of conjugating the SFB to the Annexin V may include the steps of placing the SFB into a methylene chloride solution, evaporating the methylene chloride solution to dryness using a stream of nitrogen to produce a residue, adding a solution of Annexin V to the residue, and incubating a result of the adding step.

### BRIEF DESCRIPTION OF THE DRAWINGS

FIG. 1 is an illustration of a first exemplary chemical reaction for synthesis of [¹⁸F]SFB according to a preferred embodiment of the present invention.

FIG. 2 is an illustration of a second exemplary chemical reaction for synthesis of [¹⁸F]SFB according to a preferred embodiment of the present invention.

FIG. 3 is an illustration of a third exemplary chemical reaction for synthesis of [¹⁸F]SFB according to a preferred embodiment of the present invention.

FIG. 4 is an illustration of a fourth exemplary chemical reaction for synthesis of [¹⁸F]SFB according to a preferred embodiment of the present invention.

FIG. 5 is a flow chart that illustrates a method of using F-18 labeled Annexin V to quantify apoptosis in a lesion or tumor, according to a preferred embodiment of the present invention.

### DETAILED DESCRIPTION OF THE INVENTION

There are two primary processes that lead to cell death, necrosis and apoptosis. Necrosis is caused by gross disruption of the cell membrane and is often the result of significant osmotic, mechanical, or chemical damage. Molecular identification of necrosis is not well defined due to the chaotic nature of cell death. The most common histologic feature of necrosis is perinecrotic inflammation.

Apoptosis is a genetically defined cell death that involves activation of a core enzymatic machine consisting of cysteine proteases, called caspases. In most cases, the activation of caspases represents irreversible progression to cell death. The process of apoptosis occurs during normal growth and differentiation to eliminate cells that are no longer necessary for tissue function. Tumor cells are believed to contain a block in differentiation, therefore they may be rendered more susceptible than normal cells to the induction of apoptosis.

Several studies have shown that apoptosis can be detected on an organ level by imaging. Hepatic apoptosis caused by intravenous injection of antiFas antibody in mice was detected by an increase in hepatic uptake of ^{99m}Tc-Annexin measured both by region of interest (ROI) analysis and biodistribution data. Higher ^{99m}Tc-Annexin uptake in cardiac allografts compared to control cardiac isografts in rats has also been observed. In early clinical studies, cardiac SPECT imaging with ^{99m}Tc-Annexin has been used to visualize cardiac transplant rejection. It has been reported that ^{99m}Tc-Annexin V has rapid blood clearance in humans, and it has been stated that this rapid clearance should permit diagnostic images on transplant rejection within 2 hours post-injection.

The relatively small size of tumors compared to whole organs makes detection of apoptosis in tumors by external imaging more challenging. Several research groups reported significant increases in the localization of ^{99m}Tc-Annexin V after chemotherapy in B-cell lymphomas, breast cancer tumors, and hepatomas in rodents by imaging 1-2 hours post-injection of ^{99m}Tc-Annexin V. SPECT imaging studies of Apomate™ (Theseus Imaging Corporation's kit for the preparation of ^{99m}Tc labeled Annexin V) have begun in patients with lymphomas, sarcomas, breast and lung cancers. In several studies, patients who showed increased tumor signal with Apomate™ after chemotherapy demonstrated better therapeutic outcomes than patients whose tumors demonstrated little uptake. In these studies imaging was performed at two and 24 hours post-injection of Apomate™. The sensitivity of a dedicated PET camera is 20-100 times greater than SPECT and the 4-5 mm spatial resolution of PET is superior to the 8-1 3 mm spatial resolution of SPECT; therefore, a strong case can be made that imaging cell death in tumors could best be done with PET imaging using a labeled compound such as ¹⁸F-Annexin V. Since useful images of chemotherapy induced tumor cell death were obtained at 2 hours post-injection of ^{99m}Tc-labeled Annexin V, the 110 minute half-life of ¹⁸F should be sufficiently long-lived to allow ¹⁸F labeled Annexin V to provide corresponding PET images. PET cameras can rigorously correct for the effect of gamma-ray attenuation, and thereby provide quantitative measurements of radiotracer concentration in vivo. The recent availability of high spatial resolution animal PET scanners has led to strong interest in PET ligands for rational drug development.

Referring to FIG. 5, a flow chart 500 illustrates a method of quantifying apoptosis using PET scanning images and F-18 labeled Annexin V according to a preferred embodiment of the present invention. The first step 505 is to label the Annexin V using a positron emitter, such as F-18, as further described below. The second step 510 is to inject the F-18 labeled Annexin V into a lesion or suspected tumor, or into any target cell group for which it is desired to quantify an amount of apoptosis. Then, in step 515, a PET scanner is used to produce an image of the target cell group. Finally, in step 520, the image is analyzed to determine an amount of cell death in the target cell group.

The use of radiolabeled agents to describe tumor biochemistry in vivo requires that the radiolabel be prepared with high specific activity. In other words, the ratio of radiolabeled target-binding agent to non-radiolabeled target-binding agents needs to be as high as possible. Methods used to achieve high specific activity are different when labeling proteins (e.g., Annexin), as compared to when small non-protein molecules (e.g., glucose) are labeled. When radiolabeling small non-protein molecules, high specific activity is achieved by starting with high specific activity radionuclide, using a no-carrier-added labeling reaction, followed by a chromatographic separation of labeled compound from free radionuclide and unlabeled precursor. When preparing high specific activity radiolabeled proteins, the changes in the properties of the protein caused by the radiolabel (charge, lipophilicity, polarity, size) are too small to permit a chromatographic separation of radiolabeled and non-labeled protein; therefore, specific activity is determined by the number of moles of radionuclide and protein as well as the efficiency of the labeling reaction.

High specific activity labeling with fluorine requires different methods than high specific labeling with iodine. Proteins can be radiolabeled with iodine via direct electrophilic radioiodination of tyrosine groups. With this method, the specific activity of the radiolabeled protein is controlled by the amount of protein present, because the efficiency of the labeling reaction is usually very high. Unfortunately, the fluorine equivalent (i.e., electrophilic fluorination) requires excess nonradioactive fluoride and thus can only produce low specific activity labeled protein. To prepare high specific activity fluorinated proteins, researchers have taken an approach analogous to the Bolton-Hunter method of protein radioiodination. In this approach an ¹⁸F labeled small molecule containing a protein conjugating group is synthesized and purified by chromatographic methods to produce a high specific activity prosthetic group. The ¹⁸F labeled prosthetic group is then conjugated to the desired protein. With this two-step approach, the specific activity of the labeled protein depends on the specific activity of the ¹⁸F labeled prosthetic group, the amount of protein used, and the efficiency of the conjugation reaction.

Several ¹⁸F labeled prosthetic groups appear in the literature, and are summarized in Table 2. Deciding which group to use for conjugation with Annexin V depends on many factors including yield, specific activity, speed and ease of synthesis and purification of the ¹⁸F labeled prosthetic group. In addition, the protein conjugation group on the prosthetic molecule must be matched with available reactive groups on the protein. The higher the number of reactive moieties on the protein, the more likely the conjugation reaction will proceed with high efficiency; however, the possibility of multiple conjugations per single protein molecule also increases. Although multiple conjugations would tend to increase yield, too many conjugations might reduce biologic activity due to steric blockage of the PS binding site on Annexin V.

TABLE 2
Summary of [¹⁸F]Prosthetic Groups And Their Use In Protein Labeling.

| [¹⁸F]Prosthetic | | | | Protein Labeling | | | |
|---|---|---|---|---|---|---|---|
| Group Preparation | | | | | | % Yield | |
| | # of | | % | | Reactive | (Specific | Comments |
| Group | steps | Time | Yield | Protein | Group | Activity) | — |
| 4-[¹⁸F]fluoro- | 3 | 75 min | 28%-40% | HSA | RSH | 95% | — |
| phenacyl-bromide | | | | fibrinogen | | 25-30% | |
| (FPB)¹⁷⁻¹⁹ | | | | | | | |
| 4-[¹⁸F]fluoro- | 3 | <35 min | 65% | HSA | RSH | 7% | Quoted |
| phenacyl-bromide | | | | | | | yield of |
| (FPB)¹⁷⁻¹⁹ | | | | | | | 65% not |
| | | | | | | | decay- |
| | | | | | | | corrected |
| N-succinimidyl-8- | 3 | 60 min | 25%-40% | IgG or | lysine | 47% | Mixed |
| [(4′-[¹⁸F]fluoro- | | | | F(ab′)₂ | | | cross-linked |
| benzyl) amino] | | | | | | | Byproducts |
| suberate (SFBS)²⁰ | | | | | | | |
| N-succinimidyl-4- | 3 | 100 min | 25% | F(ab′)₂ | lysine | 40-60% | DCU |
| [¹⁸F]fluorobenzoate | | | | | | (500 Ci/mmol) | precipitates, |
| (SFB)²¹ | | | | | | | clogs HPLC |
| N-succinimidyl-4- | 3 | 55 min^{a} | 34% | F(ab′)₂ | lysine | 50% (300 Ci/mmol) | DCU not |
| [¹⁸F]fluorobenzoate | | | | Unknown | | | produced; |
| (SFB)²² | | | | peptide | | | 150° C. |
| | | | | | | | reaction |
| N-succinimidyl-4- | 3 | 60 min | 65%-80% | Labeling | — | — | 95° C. |
| [¹⁸F]fluorobenzoate | | | | not done | | | reaction |
| (SFB)²³ | | | | | | | |
| N-succinimidyl-4- | 1 | 30-50 min | 16%-18% | DsFv | lysine | 40% (1500 Ci/mmol) | Fewest |
| [¹⁸F](fluoromethyl)benzoate²⁴ | | | | Transferrin | | 33% (2200 Ci/mmol) | steps but |
| | | | | Unknown | | 60% (4000 Ci/mmol) | lowest yield |
| | | | | protein | | | |

One of the earliest syntheses of an ¹⁸F labeled prosthetic group was that of 4-[¹⁸F] fluorophenacyl bromide (FPB). FPB was prepared in three steps with a total yield of 28-40% in 75 min. and attached to human serum albumin (HSA) and fibrinogen at 47° C. in 95% and 25-30% yields respectively. A quicker higher yield three step synthesis (<35 min., 65% yield) was later reported. Unfortunately, labeling of HSA at room temperature (temperature less likely to denature proteins) with FPB prepared by the latter route was much lower (7%). Higher protein labeling yields (70%) were achieved by adding more thiol groups to HSA by pretreatment with 2-iminothiolane. However, treatment with 2-iminothiolane also increased the amount of protein crosslinking (intra- and intermolecular) from the formation of non-native disulfide bonds.

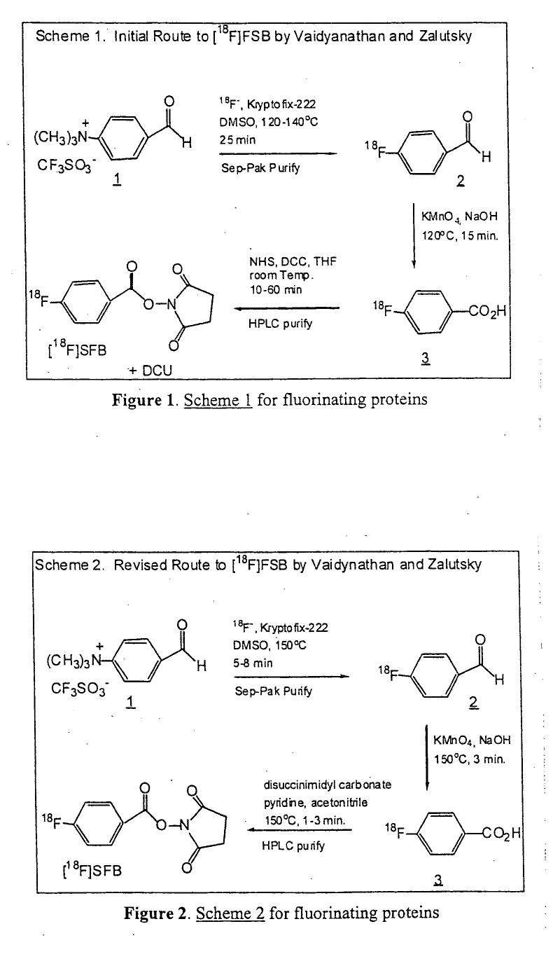

Several investigators have prepared ¹⁸F labeled prosthetic groups containing N-succinimidyl ester groups that react with lysine amines to form an amide bond. Proteins generally have more lysines than free thiol groups and therefore are better targets for labeling proteins with high efficiency. An early method utilized disuccinimidyl suberate to attach an activated ester to [¹⁸F]fluorobenzylamine; however, this can also produce a [¹⁸F]fluorobenzylamide dimer which is unreactive with protein. Referring to FIG. 1, a three-step synthesis of N-succinimidyl-4-[¹⁸F]fluorobenzoate (SFB) is outlined in Scheme 1.

Scheme 1 produces [¹⁸F]SFB in a yield of 25% in a total synthesis time of 100 minutes. In this synthesis, 4(trimethylammonium triflate)benzaldehyde (1) is reacted with [¹⁸F]fluoride and kryptofix-222 in DMSO to give 4-[¹⁸F]fluorobenzaldehyde (2) which is then oxidized to 4-[¹⁸F]fluorobenzoic acid L3), followed by formation of the activated-ester [¹⁸F]SFB. During the final step careful exclusion of air is necessary to prevent precipitation of dicyclohexyl urea (DCU) which can clog the HPLC system used in the final purification of [¹⁸F]SFB. Referring to FIG. 2, in a refinement of this route (Scheme 2), higher reaction temperatures (150° C.) were used, and disuccinimidyl carbonate was used in the final step to increase the yield of [¹⁸F]SFB to 34% in a total synthesis time of 55 minutes and eliminate the formation of DCU. [¹⁸F]SFB prepared by either route gave ¹⁸F labeled F(ab′)² fragments in a 50% yield after room temperature incubation with [¹⁸F]SFB for 15-20 minutes.

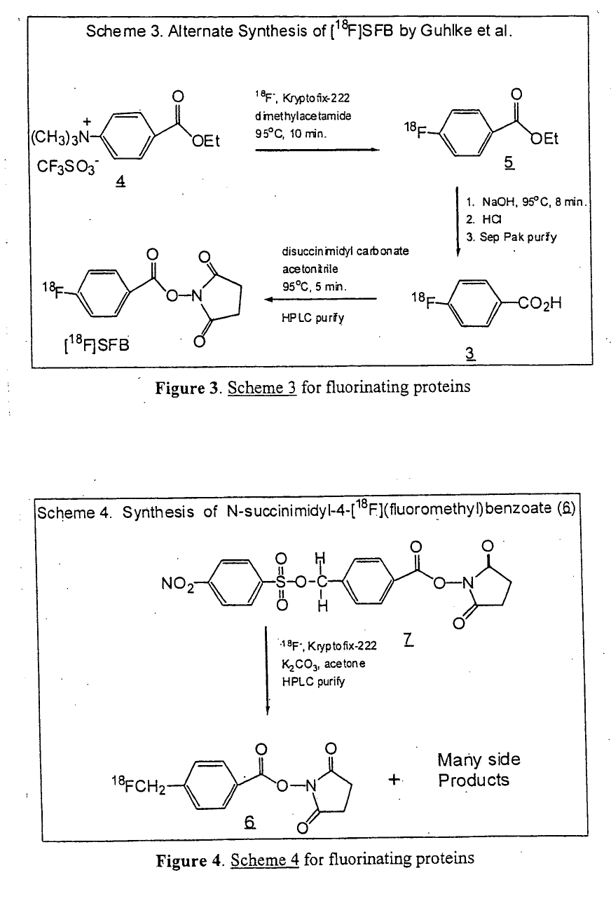

Referring to FIG. 3, an alternate three-step synthesis of [¹⁸F]SFB (Scheme 3) has been reported. Scheme 3 gives higher yields (65-80%) in a total synthesis time of one hour. In this synthesis, ethyl 4(trimethylammonium triflate)benzoate (4) is reacted with [¹⁸F]fluoride and Kryptofix-222 in dimethylacetamide to give ethyl 4-[¹⁸F]fluorobenzoate (O) which is then hydrolyzed to 4[¹⁸F]fluorobenzoic acid (3), followed by formation of the activated ester [¹⁸F]SFB as in Scheme 2.

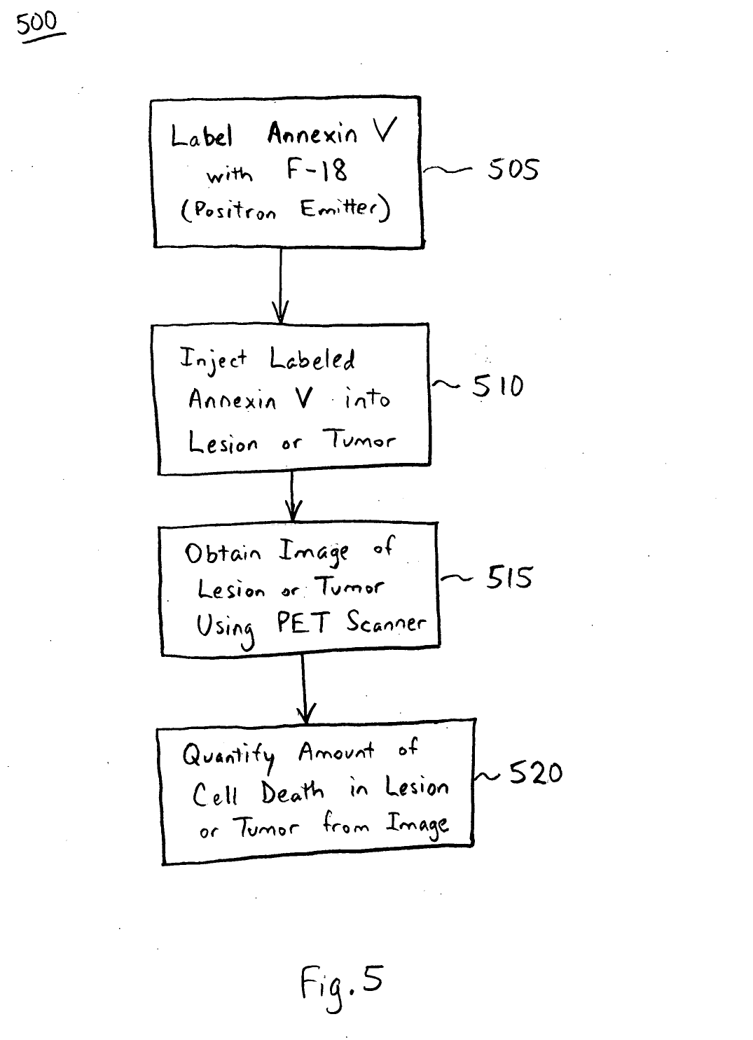

Referring to FIG. 4, a single-step synthesis of N-succinimidyl-4[¹⁸F](fluoromethyl)benzoate (7) has been reported (i.e., Scheme 4). In this synthesis N-succinimidyl 4-[(nitrobenzenesulfonyl)oxymethyl]benzoate (Z) is reacted with [¹⁸F]fluoride and Kryptofix-222 in acetone for 5 minutes and then purified by HPLC. While this one step synthesis is convenient, the yields of 6 are low (10-15%) and a careful HPLC purification is necessary to remove all the side products.

An exemplary synthesis of no-carrier-added (n.c.a.) N-Succinimidyl 4-[¹⁸F]Fluorobenzoate ([¹⁸F]SFB) has been carried out as described below: The aqueous [¹⁸F]fluoride solution is placed in a 13×100 mm borosilicate tube, 8 μL of IM potassium carbonate is added, and the tube is placed in a 95° C. oil bath. Water is evaporated under a stream of nitrogen until the volume is reduced to 50-100 μL. The radioactivity is counted and the time is recorded as the starting time of the synthesis. Then the aqueous solution of ¹⁸F— is added to a Reactavial containing 500 μL dry acetonitrile, 5.0 mg Krytofix-222, and 8 μL of 1M potassium carbonate. This mixture is evaporated to dryness at 95° C. under a stream of nitrogen. Additional dry acetonitrile (300 μL) is added to the vial and the azeotropic distillation is repeated. The addition of acetonitrile and evaporation under nitrogen is then repeated twice more. To the dry residue is added 10 mg of ethyl 4-(trimethylammonium triflate) benzoate dissolved in 250 μL anhydrous dimethyl acetamide, followed by heating at 150° C. for 10 minutes. The next step, hydrolysis of the ethyl ester group of ethyl 4-[¹⁸F]fluorobenzoate to 4-[¹⁸F]fluorobenzoic acid, is accomplished by the addition of 500 μL of 1 M NaOH and stirring for 8 minutes at 95° C. The reaction is then acidified with 650 μL of 1 M HCl and diluted with water to a final volume of 10 mL. The solution is drawn into a syringe with a luer lock fitting and passed through an activated C-18 Sep-Pak. Polar material is removed from the column by elution with 2.0 ml 0.01M HCl. The Sep-Pak column, still retaining 4-[¹⁸F]fluorobenzoic acid, is blown dry with a stream of nitrogen and the 4-[¹⁸F]fluorobenzoic acid (3) is eluted with 2.5 mL acetonitrile. The decay corrected yield at this point may range from 56-80%. When this procedure to prepare N-Succinimidyl 4-[¹⁸F]Fluorobenzoate ([¹⁸F]SFB) is followed by the addition of 10 mL of a 20% solution of tetrabutylammonium hydroxide to the acetonitrile solution of 4-[¹⁸F]fluorobenzoic acid, followed by evaporation to dryness at 95° C. under a stream of nitrogen and drying by azeotropic distillation with three additions of 400 μL acetonitrile, followed by the addition of a solution of 15 mg bis-N-hydroxysuccinimidyl carbonate in 300 μL acetonitrile to the dry residue, sealing the vial and heating the vial at 150° C. for 8 minutes, HPLC analysis may show an unsymmetrical peak. Further resolution of this peak by increasing the percentage of water in the HPLC eluate may show that as much as 50% of applied radioactivity eluting from the HPLC column elutes in a broad peak (not [¹⁸F]SFB) prior to the elution of the desired [¹⁸F]SFB peak. Acetonitrile/water or methanol/water may be used as the chromatography solvent. In general, acetonitrile/water mixtures give better resolution with reverse phase C18 columns than does methanol/water. Also note that as shown in Table 2, [¹⁸F]SFB prepared by this method has not ever been used to conjugate any proteins.

To improve the yield of [¹⁸F]SFB, the synthesis of [¹⁸F]SFB from 4-[¹⁸F]fluorobenzoate has been performed using another reported procedure. In this method, the acetonitrile solution of 4-[¹⁸F]fluorobenzoate that is eluted from the Sep-Pak is transferred to a round bottom flask and evaporated on a rotary evaporator using reduced pressure from a water aspirator and a room temperature heating bath. The residue is dried by multiple additions of either acetonitrile or acetone followed by evaporation on the rotary evaporator. The residue is reconstituted in acetonitrile, transferred to a Reactavial, and evaporated to about 50 μL. To the Reactavial is added 100 μL of a 0.1M solution of pyridine in acetonitrile and 100 μL of a 0.1M solution of disuccinimidylcarbonate in acetonitrile. The vial is sealed and heated at 150° C. for 6-8 minutes, cooled and purified by HPLC. This method may be modified by replacing the pyridine, which acts both as a base and as an acylation catalyst, with dimethylaminopyridine, which is a better acylation catalyst. To the residue of 4-[¹⁸F]fluorobenzoate is added 50 μL of a 0.1M solution of dimethylaminopyridine in acetonitrile and 200 μL of a 0.1M solution of disuccinimidylcarbonate in acetonitrile. The vial is sealed and heated at 150° C. for 6-8 minutes, cooled and 700 μL water added to make the acetonitrile/water ratio similar to the HPLC eluate used to purify the [¹⁸F[SFB. The addition of water causes a precipitate to form, and the suspension is transferred to a microfuge tube and centrifuged for three minutes to settle the solid. The supernatant is removed and injected onto an radio-HPLC fitted with a Delta-Pac C18, 3 micron, 3.9×150 mm column (Waters), and a variable wavelength UV detector set for 236 nm and eluted with a solution consisting of 80% water/20% acetonitrile +0.1% glacial acetic acid at a flow of 1.2 mL/min. On this system the retention time of [¹⁸F]SFB is approximately 14 minutes, and the decay corrected yield of [¹⁸F]SFB may range from approximately 52-55% in 2-3 hours preparation time.

Once the prosthetic group is synthesized and purified, it is then conjugated to Annexin V. In an exemplary preparation for the conjugation, the [¹⁸F]SFB is dissolved in the HPLC eluate (80% water/20% acetonitrile +0.1% glacial acetic acid), then diluted to a volume of 10 mL with water, drawn into a syringe and loaded onto an activated Waters Sep-Pak. The Sep-Pak is blown dry using a stream of nitrogen, and the retained [¹⁸F]SFB eluted with 2.0 mL methylene chloride.

The following exemplary conjugation of [¹⁸F]SFB to Annexin V utilizes conditions previously reported for the conjugation of [¹⁸F]SFB to antibody fragments. The methylene chloride solution of [¹⁸F]SFB is added to a 1.5 mL microfug tube and evaporated to dryness under a stream of nitrogen. To the residue in the tube is added 25-100 μL of a solution of Annexin V (5 μg/μL) in 0.1 M borate buffer, pH 8.5. This is then incubated for 20 minutes. The reaction was diluted to 100 μL with 0.1 M phosphate buffer, pH 7.4, and injected onto a TSK-2000 size exclusion column and eluted with 0.1M phosphate buffer, pH 7.4 at a flow rate of 1.0 mL/min. The retention time of Annexin V is 10 minutes. See Table 1 below for a tabulation of experimental results of conjugating [¹⁸F]SFB to Annexin V.

TABLE 1
Results:

| Rxt. | | | Decay corrected |
|---|---|---|---|
| Volume | Annexin V conc. | Annexin V used | yield of ¹⁸F-Annexin |
| 100 μL | 5 μg/μL | 500 μg | 70% |
| 100 μL | 2.5 μg/μL | 250 μg | 57% |
| 100 μL | 1.25 μg/μL | 125 μg | 38% |
| 50 μL | 5 μg/μL | 250 μg | 69% |
| 25 μL | 5 μg/μL | 125 μg | 64% |
| 25 μL* | 5 μg/μL | 125 μg | 49% |

*Entire 2.0 mL solution of [¹⁸F]SFB concentrated for this reaction.

As shown in Table 1, the highest experimental radiolabeling yields occur using an Annexin V concentration of 5 μg/μL. This requires that to produce high specific activity ¹⁸F-Annexin V, the amount of Annexin V and thus the reaction volume must be small. Good yields of ¹⁸F-Annexin V can be produced in a 25 μL volume (125 μg); however, when starting with a large volume of [¹⁸F]SFB in methylene chloride, yields can be reduced because of the difficulty in concentrating a large volume of [¹⁸F]SFB into a small area where it can be readily dissolved in 25 μL.

The binding activity of [¹⁸F]SFB-Annexin V to cells expressing phosphatidylserine (PS) has been performed using red blood cells (RBCs). Normal RBCs have very few binding sites for Annexin; however, RBCs from commercial preserved whole blood (e.g., Coulter 4 Cplus Normal Control) have high levels of exposed PS. [¹⁸F]SFB-Annexin V at a concentration of 12 nmol/L final concentration is added to each of two tubes containing 1 mL of buffer HNKGB (10 mM HEPES-Na pH 7.4, 136 mM NaCl, 2.7 MM KCl, 5 mM glucose, and 1 mg/mL BSA) plus 2.5 mM CaCl₂. To one tube is added 4.2×10⁸ RBCs. Both tubes are incubated at room temperature for 30 minutes, then centrifuged for 3 minutes at 2000×g. The supernatants are removed and both the supernatants from both tubes and cells remaining in the single tube are counted. The percentage of radioactivity bound to the cells is calculated from 100× (1-(supernatant counts in the presence of cells)/supernatant counts in the absence of cells).

Results:

Percent binding to cells were 50-52%, n=4;

Reported values for ^{99m}Tc-Annexin V are 75-85%.

While the present invention has been described with respect to what is presently considered to be the preferred embodiments, it is to be understood that the invention is not limited to the disclosed embodiments. To the contrary, the invention is intended to cover various modifications and equivalent arrangements included within the spirit and scope of the appended claims. The scope of the following claims is to be accorded the broadest interpretation so as to encompass all such modifications and equivalent structures and functions.

The following publications are incorporated by reference herein:
1. Martin S J, Reutelingsperger C P, McGahon A J, Rader J A, van Schie R C, LaFace D M, Green D R. Early redistribution of plasma membrane phosphatidylserine is a general feature of apoptosis regardless of the initiating stimulus: inhibition by overexpression of Bcl-2 and Abl. J Exp Med. 1995; 182:1545-56.
2. Koopman G, Reutelingsperger C P, Kuijten G A, Keehnen R M, Pals S T, van Oers M H. Annex V for flow cytometric detection of phosphatidylserine expression on. B cells undergoing apoptosis. Blood. 1994; 84:1415-20.
3. Gelmon K A, Eisenhauer E A, Harris A L, Ratain M J, Workman P. Anticancer agents targeting signaling molecules and cancer cell environment: challenges for drug development? J Cancer lnst. 1999; 91:1281-7.
4. Harmon B V, Corder A M, Collins R J, Gobe G C, Allen J, Allan D J, Kerr J F. Cell death induced in a murine mastocytoma by 4247 degrees C. heating in vitro: evidence that the form of death changes from apoptosis to necrosis above a critical heat load. Int J Radiat Biol. 1990; 58:845-58
5. Ogasawara J, Watanabe-Fukunaga R, Adachi M, Matsuzawa A, Kasugai T, Kitamura Y, Itoh N, Suda T. Nagata S. Lethal effect of the anti-Fas antibody in mice [published erratum appears in Nature 1993 Oct. 7; 365(6446):568]. Nature. 1993; 364:806-9.
6. Ohtsuki K, Akashi K, Aoka Y, Blankenberg F G, Kopiwoda S, Tait J F, Strauss H W. Technetium-99m HYNIC-annexin V: a potential radiopharmaceutical for the in-vivo detection of apoptosis. Eur J Nucl Med. 199; 26:1251-8.
7. Blankenberg F G, Katsikis P D, Tait J F, Davis R E, Naumovski L, Ohtsuki K, Kopiwoda S, Abrams M J, Strauss H W. Imaging of apoptosis (programmed cell death) with 99m Tc annexin V. J Nucl Med. 1999; 40:184-91.
8. Blankenberg F D, Katsikis P D, Tait J F, Davis R E, Naumovski L, Ohtsuki K, Kopiwoda S, Abrams M J, Darkes M, Robbins R C, Maecker H T, Strauss H W. In vivo detection and imaging of phosphatidylserine expression during programmed cell death. Proc Natl Acad Sci USA. 1998; 95:6349-54.
9. Green A M, Steinmetz, N. D., A new kit for the preparation of technetium 99mTc recombinant human annexin V for apoptosis imaging-biodistribution, dosimetry, and initial imaging experence. J. Nucl. Med. 2000; 41:6P abstract.
10. Acio E, Fitzpatrick J M, Samuels L E, Guerraty A, Fyfe B, Narula N, Tomaszewski J E, Snyder G, Kelly C, and Narula J. Phase-1 99mTc-Annexin-V imaging study for noninvasive detection of apoptosis during allograft rejection in heart transplants. Journal of Nuclear Medicine. 2000; 41:127P:
11. Strass H W K, Hunt S, Robbins R, Stafford-Cecil S, Tait J F, and Blankenberg F G. Blood clearance of ^{99m}Tc-N₂S₂Annexin V in human subjects. Journal of Nuclear Medicine. 2000; 41: 149P.
12. Yang D J Z S, Azhdarina A, Yu D F, Kalimi S K, Kim E E, Podoloff D A. In vivo and in vitro measurement of apoptosis in breast cancer cells using ^{99m}Tc-EC-annexin V. Journal of Nuclear Medicine. 2001; 42:83P.
13. Mochizuki T KY, Zhao S, Hikosaka K, Tsukamoto E, Strass W H, Blankenberg F, Tait J, Tamaki N. Detection of tumor response following a single dose of chemotherapy with ^{99m}Tc-annexin V. Journal of Nuclear Medicine. 2001:84P.
14. Belhocine T, Hustinx, R., Jersulem, G., Fassotte, M., Duysinx, B., Quaden, C., Rigo, P. 99mTc RHAnnexinV (Apomate™) as a marker of apoptosis resulting from chemotherapy: preliminary results. J. Nucl. Med. 2000; 41:263P.
15. Steinmetz N D S A, Woolfenden J M, Heidendal G A, Hofstra L, Thimister P, Belhocine T Z, Rigo P, Green A M. Imaged cell death in untreated and treated human tumors with 99mTc annexin V prepared using the Apomate kit. Journal of Nuclear Medicine. 2001; 42:83P.
16. Hebel M S, Kirk, K. L., Cohen, L. A., Labroo, V. M. First direct fluorination of tyrosine-containing biological active peptides. Tetrahedron Letters. 1990; 31:619.
17. Kilboum M R, Dence C S, Welch M J, Mathias C J. Fluorine-18 labeling of proteins. J Nucl Med. 1987; 28:462-70.
18. Dence C S, McCarthy T J, Welch M J. Improved synthesis of [18F]4-fluorophenacyl bromide: the use of polymer supported perbromide. Appl Radiat Isot. 1993; 44:981-3.
19. Downer J B, McCarthy T J, Edwards W B, Anderson C J, Welch M J. Reactivity of p-[18F]fluorophenacyl bromide for radiolabeling of proteins and peptides. Appl Radiat Isot. 1997; 48:907-16.
20. Garg P K, Garg S, Zalutsky M R. Fluorine-18 labeling of monoclonal antibodies and fragments with preservation of immunoreactivity. Bioconjug Chem. 1991; 2:44-9.
21. Vaidyanathan G, Zalutsky M R. Labeling proteins with fluorine-18 using N-succinimidyl 4[18F]fluorobenzoate. Int J Rad Appl Instrum B. 1992; 19:275-81.
22. Vaidyanathan G, Zalutsky M R. Improved synthesis of N-succmunidyl 4-[18F]fluorobenzoate and its application to the labeling of a monoclonal antibody fragment. Bioconjug Chem. 1994; 5:352-6.
23. Guhlke S, Coenen, H. H., Stocklin, G. Fluoroacylation agents based on small n.c.a[18F]fluorocarboxylic acids. Appl. Radiat. Isot 1994; 45:715-727.
24. Lang L, Eckelman W C. One-step synthesis of 18F labeled [18F]-N-succinimidyl 4(fluoromethyl)benzoate for protein labeling. Appl Radiat Isot 1994; 45:1155-63.
25. Lang L, Eckelman W C. Labeling proteins at high specific activity using N-succinimidyl 4[18F](fluoromethyl) benzoate. Appl Radiat Isot. 1997; 48:169-73.
26. Tait J F, Gibson D. Measurement of membrane phospholipid asymmetry in normal and sickle-cell erythrocytes by means of annexin V binding. J Lab Clin Med. 1994; 123:741-8.
27. Tait J F, Engelhardt S, Smith C, Fujikawa K. Prourokinase-annexin V chimeras. Construction, expression, and characterization of recombinant proteins. J Biol. Chem. 1995; 270:21594-9.

What is claimed is:

1. A method for noninvasive measurement of apoptosis, the method comprising the steps of:
labeling Annexin V with a positron emitter;
administering the labeled Annexin V to a patient containing a target cell group;
obtaining an image of the target cell group using a positron emission tomography scanner; and
evaluating the image to determine an amount of cell death within the target cell group.

2. The method of claim 1, wherein the target cell group is a lesion.

3. The method of claim 1, wherein the target cell group is a suspected tumor.

4. The method of claim 1, wherein the target cell group is normal tissue.

5. The method of claim 1, wherein the positron emitter comprises F-18.

6. The method of claim 5, wherein the step of labeling Annexin V with F-18 comprises the steps of:
selecting an F-18 labeled small molecule containing a protein conjugating group;
synthesizing and purifying the selected molecule;
producing a high specific activity prosthetic group as a result of the synthesizing and purifying step; and
conjugating the prosthetic group to the Annexin V.

7. The method of claim 6, wherein the produced prosthetic group comprises 4-[¹⁸F]fluorophenacyl-bromide (FPB).

8. The method of claim 6, wherein the produced prosthetic group comprises N-succinimidyl-8-[(4′-[¹⁸F]fluorobenzyl)amino] suberate (SFBS).

9. The method of claim 6, wherein the produced prosthetic group comprises N-succinimidyl-4-[¹⁸F]fluorobenzoate (SFB).

10. The method of claim 9, wherein the step of synthesizing and purifying includes the steps of:
reacting 4(trimethylammonium triflate) benzaldehyde with [¹⁸F]fluoride and kryptofix-222 in DMSO to produce 4-[¹⁸F]fluorobenzaldehyde;
oxidizing the 4-[¹⁸F]fluorobenzaldehyde to produce 4-[¹⁸F]fluorobenzoic acid;
treating the 4-[¹⁸F]fluorobenzoic acid with NHS, DCC, and THF at substantially room temperature to form SFB; and
using HPLC to purify the SFB.

11. The method of claim 9, wherein the step of synthesizing and purifying includes the steps of:
reacting 4(trimethylammonium triflate) benzaldehyde with [¹⁸F]fluoride and kryptofix-222 in DMSO to produce 4-[¹⁸F]fluorobenzaldehyde;
oxidizing the 4-[¹⁸F]fluorobenzaldehyde to produce 4-[¹⁸F]fluorobenzoic acid;
treating the 4-[¹⁸F]fluorobenzoic acid with disuccinimidyl carbonate and acetonitrile at substantially 150° C. to form SFB; and
using HPLC to purify the SFB.

12. The method of claim 9, wherein the step of synthesizing and purifying includes the steps of:
reacting ethyl 4(trimethylammonium triflate) benzoate with [¹⁸F]fluoride and kryptofix-222 in dimethylacetamide to produce ethyl 4-[¹⁸F]fluorobenzoate;
hydrolyzing the 4-[⁸F]fluorobenzoate to produce 4-[¹⁸F]fluorobenzoic acid;
treating the 4-[¹⁸F]fluorobenzoic acid with disuccinimidyl carbonate and acetonitrile at substantially 150° C. to form SFB; and
using HPLC to purify the SFB.

13. The method of claim 9, wherein the step of synthesizing and purifying includes the steps of:
reacting N-succinimidyl-4-[(nitrobenzenesulfonyl)oxymethyl]benzoate with [¹⁸F]fluoride and kryptofix-222 in acetone to produce SFB; and
using HPLC to purify the SFB.

14. The method of claim 9, wherein the step of synthesizing and purifying includes the steps of:
placing an aqueous [¹⁸F]fluoride solution in a borosilicate tube;
adding substantially 8 μL of IM potassium carbonate;
placing the tube in a substantially 95° C. oil bath;
evaporating water under a stream of nitrogen until a volume is reduced to substantially 50-100 μL;
counting a radioactivity;
recording a starting time;
adding the aqueous solution of [¹⁸F] to a vial containing substantially 500 μL dry acetonitrile, substantially 5.0 mg Krytofix-222, and substantially 8 μL of 1M potassium carbonate;
evaporating a resulting mixture to dryness at substantially 95° C. under a stream of nitrogen;
adding substantially 300 μL of dry acetonitrile to the vial;
re-evaporating a resulting mixture to dryness at substantially 95° C. under a stream of nitrogen;
repeating the third adding step and the re-evaporating step twice each to produce a residue;
adding substantially 10 mg of ethyl 4-(trimethylammonium triflate) benzoate dissolved in 250 μL anhydrous dimethyl acetamide;
heating at substantially 150° C. for substantially 10 minutes;
adding substantially 500 μL of 1 M NaOH;
stirring for substantially 8 minutes at substantially 95° C.;
acidifying with substantially 650 μL of 1 M HCl;
diluting with water to produce a solution having a volume of substantially 10 mL;
drawing the produced solution into a syringe with a luer lock fitting;
passing through an activated C-18 Sep-Pak;
removing polar material by elution with substantially 2.0 mL 0.01M HCl, wherein 4-[¹⁸F]fluorobenzoic acid is retained;
blowing dry with a stream of nitrogen;
eluting the 4-[¹⁸F]fluorobenzoic acid with substantially 2.5 mL acetonitrile to form a solution of 4-[¹⁸F]fluorobenzoate;
transferring the solution of 4-[¹⁸F]fluorobenzoate to a round bottom flask;
evaporating on a rotary evaporator using reduced pressure from a water aspirator and a room temperature heating bath to form a residue;
drying the formed residue by multiple additions of either acetonitrile or acetone followed by evaporation on the rotary evaporator;
reconstituting the residue in acetonitrile;
transferring to a vial;
evaporating to substantially 50 μL;
adding substantially 50 μL of a 0.1M solution of dimethylaminopyridine in acetonitrile and 200 μL of a 0.1M solution of disuccinimidylcarbonate in acetonitrile to the vial;
sealing the vial;
heating the vial at substantially 1 50° C. for substantially 6-8 minutes;
cooling;
adding substantially 700 μL of water, wherein a precipitate is formed within a suspension;
transferring the suspension to a microfuge tube;
centrifuging for substantially three minutes;
removing a supernatant;
injecting the supernatant onto an radio-HPLC fitted with a Delta-Pac C18, 3 micron, 3.9×150 mm column (Waters), and a variable wavelength UV detector set for substantially 236 nm and eluted with a solution consisting substantially of 80% water/20% acetonitrile +0.1% glacial acetic acid at a flow of substantially 1.2-mL/min.

15. The method of claim 9, wherein the step of conjugating the SFB to the Annexin V includes the steps of:
placing the SFB into a methylene chloride solution;
evaporating the methylene chloride solution to dryness using a stream of nitrogen to produce a residue;
adding a solution of Annexin V to the residue; and
incubating a result of the adding step.

16. A method of prediction of cell death, the method comprising the steps of:
applying a method of killing to a target cell group;
labeling Annexin V with a positron emitter;
administering the labeled Annexin V to a patient containing the target cell group;
obtaining an image of the target cell group using a positron emission tomography scanner; and
evaluating the image to determine an amount of cell death within the target cell group.
